(19) United States
(12) Patent Application Publication
(10) Pub. No.: US 2004/0259188 A1
(43) Pub. Date: Dec. 23, 2004

(54) Method and system for processing bio-contaminated articles

(76) Inventors: Aaron A. Rosenblatt, New York, NY (US); Thomas E. McWhorter, Allentown, PA (US)

Correspondence Address: RATNERPRESTIA, P O BOX 980, VALLEY FORGE, PA, 19482-0980, US

(21) Appl. No.: 10/494,230

(22) PCT Filed: Nov. 4, 2002
(86) PCT No.: PCT/US02/35519

Related U.S. Application Data
(60) Provisional application No. 60/338,981, filed on Nov. 5, 2001.

(51) Int. Cl. C12Q 1/22; A01N 1/00

(52) U.S. Cl. 435/31; 422/37

(57) ABSTRACT

Method and system for decontaminating letters and packages deposited with, collected by and forwarded by, e.g. the US Postal Service. Letters and packages are collected in a substantially microorganism impermeable but gas permeable receptacle which can be sealed and placed in a sterilizing gas atmosphere consisting of chlorine dioxide a diluent, and optionally water vapor.

### BACKGROUND OF THE INVENTION

The present invention relates to decontamination of bio-contaminated articles.

The mail system, including operations by governmental and private concerns, has from time to time been used nefariously as a vehicle for delivery of biological pathogens intended to cause harm to the recipients of the delivered pathogen-containing item.

In October 2001, for example, Anthrax spores were sent by mail to US Senators, broadcast firms, and newspaper publishers, among others. The Anthrax spores caused facilities contamination, illness and death, both at the locations of the addressees to which the spore-containing letters were delivered, and also to postal workers and others who were proximate to spores released during transport and processing of the pathogen-containing mail. The contamination also extended to persons who were remote to the Anthrax-containing letters, most likely because of multi-generation cross contamination of other mail. That is, the victims received letters that were in contact with other letters that were contaminated during processing and handling of the original Anthrax-containing missives.

Typically, public and private mail and parcel delivery systems have three basic operations: (1) collection, (2) processing, and (3) delivery. Each of these three broad categories is made up of numerous component functions, and the systems can be quite extensive.

For example, the United States Postal Service (USPS) has approximately 800,000 employees. Mail is collected, principally by letter carriers, from approximately 315,000 USPS mailboxes plus from bulk mail shippers and others. The collected mail typically is put into canvas bags, and consolidated at 38,000 postal stations (e.g., local post offices). From these postal stations, most of the mail is transported to and further consolidated at approximately 250 central processing/distribution facilities. A portion of the mail, especially in large US cities, may be transported to about 150 intermediate processing facilities, prior to being sent to a central processing/distribution site. After processing, which includes sorting and routing, the mail is distributed back to local postal stations for delivery to addressees.

Various methods have been proposed for prophylactic processing of the mail, the methods intended to destroy pathogens that might be contained therein. Processes proposed include centralized irradiation of the mail by well-known techniques, such as methods that use electron beam irradiation, and methods that use gamma radiation emanating from a radioactive source, such as radioactive cobalt. These methods, some of which are widely used in the sterilization of medical devices, have certain technical and practical limitations for decontamination of mail. For example, electron beam radiation is known to have limited ability to penetrate articles being treated and is energy intensive. Gamma radiation requires the presence of a radioactive material, which necessitates extraordinary safety and security precautions. Both methods typically require massive, costly installations suitable only to large centralized facilities.

Centralization of mail-decontamination processes, for example, using electron beam irradiation, theoretically can protect the “back end” of the mail system, preventing infectious pathogens (such as Anthrax) from being released post-decontamination, during distribution and delivery. However, such a centralized approach leaves unprotected the “front end” of the system, comprising collection and consolidation at processing/distribution centers. Such centralized treatment approaches leave especially vulnerable and unprotected the letter carriers and other personnel who collect the mail and transport it to the central processing facilities. It also leaves the entire system open to extensive cross-contamination, which diminishes substantially the value of prophylactic treatment that is limited to high-risk portions of the system, such as mail addressed to governmental agencies, news media and the like.

Gas sterilization is well known in the medical device and pharmaceutical industries where it has been employed to treat packaged medical devices and, to a limited extent, isolators (i.e., “clean rooms). Microbial inactivation with gaseous chemosterilants is a function of several parameters, including gas concentration, time, temperature and relative humidity. It is a preferred practice in the medical device manufacturing industry to develop knowledge of and document the set of interrelated parameters required to achieve a desired level of “kill” for a particular target organism, and to then assume that a device has been sterilized if it can be shown that the device has been subjected to conditions which at least meet said parameters.

Typically, there is some trade-off between the critical gas-sterilization parameters of time, relative humidity, temperature and gas concentration, but the relationships are not necessarily linear. It is customary to use a non-pathogenic surrogate organism to model the expected behavior of a highly pathogenic one. Bacillus subtilus v. niger is widely recognized as an appropriate surrogate for chemo-sterilization resistant organisms, such as Anthrax, and has been used to develop and validate medical-sterilization regimes. Because medical devices are substantially contamination-free prior to sterilization, the standard for assuring sterility is a cycle that reliably achieves 6-logs of “kill”. However, a sterilant's ability to achieve a certain level of kill, e.g., 6 logs, does not necessarily mean that a higher concentration of the sterilant, or its application for a longer period of time, will be able to achieve higher levels of kill.

When pathogens are intended for use as biological warfare agents (BWA), as in recent cases of mail-borne pathogen, the pathogens may be specially prepared (“weaponized”) so that they can aerosolize and be inhaled by victims. Weaponized spores, such as those that cause the particularly deadly “inhalation Anthrax”, have several distinguishing characteristics: (1) They are small, reportedly on the order of 1-3 microns in size. This facilitates their easy dispersion and ready entry deep into victims' lungs. (2) The particles remain discreet, i.e., they don't “clump” together, and are able to be aerosolized; and (3) in at least some cases, there is a high concentration of spores per unit of material. (The weaponized Anthrax used in the cases of recent contamination reportedly contained 10⁸ to 10¹² spores per gram.).

Weaponizing may involve several steps, including drying and milling spores to the desired size. However, several factors, including the natural hygroscopicity of spores and electrostatic surface charge that may be associated with milling fine particles, may cause the finely milled spores to clump together. In order to keep weaponized spores finely divided and to prevent “clumping”, they may be treated in various ways. Such processes help prevent clumping and facilitate aerosolization. However, these procedures also make much more difficult the inactivation of the dry, fine-milled spores. Procedures that are sufficient to kill “natural” spores are not necessarily effective against “weaponized” spores.

### BRIEF SUMMARY OF THE INVENTION

In order to provide an effective method and system for decontaminating mail, it has been discovered that a gaseous mixture of chlorine dioxide and a diluent can be used in combination with a substantially microorganism-impermeable but gas permeable collection container for the mail.

Therefore, in one aspect the present invention is a method for decontaminating articles such as letters and packages deposited with, collected by and forwarded by one of the federal postal service or a private delivery service comprising the steps of; collecting the articles in a receptacle substantially impermeable to micro-organisms but at least partially gas permeable and adapted to be sealed and transported, and exposing the receptacle to an atmosphere consisting of a mixture of a sterilizing gas and a diluent, the mixture permeable to the container, with the sterilizing gas present in an amount to decontaminate the articles.

In another aspect the present invention is a system for collecting articles such as letters and packages deposited with, collected by and forwarded by one of the federal postal service or private delivery service companies comprising in combination; a receptacle adapted to receive the articles, the receptacle being substantially impermeable to micro-organisms and permeable to a sterilizing gas, and means to seal the receptacle in order to contain the articles within the receptacle for transport.

In yet another aspect the present invention is a method for decontaminating bio-contaminated articles comprising the steps of placing the articles in a receptacle that is one of substantially impermeable to micro-organisms but permeable to gas or substantially impermeable to both micro-organisms and gas; and exposing the articles to an atmosphere consisting of a mixture of a sterilizing gas and a diluent, the mixture containing an effective amount of sterilizing gas to decontaminate the bio-contaminated articles.

### DETAILED DESCRIPTION OF THE INVENTION

The present invention provides a system and means for safely collecting, transporting, processing and delivering the mail. The method and system according to the invention are most advantageously undertaken as an integral approach, but various components may also be deployed individually, to some benefit. Generally, the present invention comprises one or more of: (1) a removable microbiological-containment device, e.g., a mailbox liner/transport/treatment module, into which collected mail is deposited at the beginning of the mail collection cycle; (2) transport of the mail-containing transport/treatment module (device) to a location for application of a gaseous decontamination procedure to the contents of the device while the contents are still contained in the transport/treatment module, and (3) post-decontamination processing and delivery of the mail. Sub-steps, such as microbiologically-contained transfer from the device of step (1) to a separate microbiologically-contained transport device or from the collection/transport device of step (1) to the decontamination procedure of step (2), are consistent with and included within the scope of the present invention.

Gaseous decontaminating agents, such as chlorine dioxide, may be advantageously applied to mail, since gas molecules can diffuse thoroughly through cracks, crevices and pores in mail and reach any surface that might have been reached by the target pathogen(s).

Chlorine dioxide gas is well known to kill resistant pathogenic organisms, such as bacillus subtilus v. niger, that are commonly used surrogates for pathogenic organisms, such as Anthrax spores. The extent of microbial kill by chlorine dioxide, as with other chemosterilants, is a function of several factors, including contact time, gas concentration, temperature and relative humidity of the treating atmosphere. In the case of Anthrax spores the chlorine dioxide should be present in a range of between 500 and 10,000 parts per million by volume of the mixture. For other micro-organisms the concentration will vary and a lower level maybe adequate.

Chlorine dioxide generated by some methods, such as acidification of sodium chlorite solution with HCl, contains chlorine as an impurity. The solutions used in such methods may also be highly acidic. If the means of generating chlorine dioxide gas involves starting with a solution-based method and the “sparging” of the gas product from the liquid, acid vapor as well as chlorine gas will be contained in the chlorine dioxide product. Chlorine, especially in the presence of humidity, is highly corrosive to metals and incompatible with many non-metallic materials. Chlorine also interferes, giving “false positives”, with many analytical techniques used to measure chlorine dioxide gas. Acid vapors are also corrosive. Substantially chlorine-free chlorine dioxide can be produced by certain methods, such as in the Gas:Solid method or chlorine can be selectively removed from the chlorine dioxide by any of several methods, prior to use of the chlorine dioxide as a decontaminant.

In deploying chlorine dioxide gas for mail decontamination, it is essential to use a sufficient amount of gas for a sufficient length of time to assure that pathogens have been killed. In addition control of the temperature and relative humidity of the atmosphere under which the mail is being decontaminated must be maintained. At the same time, it is advantageous to minimize deleterious effects on materials comprising mail being treated. It is also advantageous to process the mail as quickly as possible.

Chlorine dioxide is subject to photolytic decomposition, under which it breaks down to chlorine and oxygen. In order to preserve the decontaminating ability of the chlorine dioxide gas, and to avoid the deleterious effects of chlorine gas, it is therefore necessary to protect chlorine dioxide from light, especially from ultra-violet light.

Microbial inactivation with gaseous chemosterilants is well known to be a function of several parameters, including gas concentration, time, temperature and relative humidity. It is a preferred practice in the medical device manufacturing industry to develop knowledge of and document the set of inter-related parameters required to achieve a desired level of “kill”, and to then assume that a device has been sterilized if it can be shown that the device has been subjected to conditions which at least meet said parameters. This allows for a quantitative, measurable basis for the device to be released as “sterile”, without relying on the time-consuming and somewhat qualitative culturing and testing of biological indicators that may be subject to errors due to sampling.

The following examples will serve to illustrate but not exhaustively describe the present invention.

### EXAMPLE 1

(1) A spore-impermeable, gas-permeable removable mailbox liner-transport/treatment module, e.g., a bag fabricated from a spun-bonded polyolefin, is deployed in USPS mailboxes to receive deposited mail. One type of spun-bonded polyolefin is sold by the DuPont Company under the name Tyvek™.

(2) On opening the mailbox, the module is manually or automatically sealed, such that any spores that might be in the articles of mail are safely contained in the transport/treatment module liner or bag. The contents of the module may at this point be humidified, e.g., by introduction of water vapor into the module.

(3) The sealed module is transported to a processing facility where the contents may first be exposed to a pre-humidification step, and where they are exposed to sterilizing concentrations of a gas, such as chlorine dioxide, which gas can penetrate the gas-permeable module and decontaminate the contents thereof while maintaining a barrier against pathogens which may be contained therein. The humidification step may also be carried out simultaneously with the sterilizing gas-exposure step.

(4) The contents of the module are purged or allowed to “off-gas”, so that any residual sterilizing gas which may have been sorbed by the mail has diminished to levels at which human exposure is acceptable.

(5) After a decontamination/off-gassing procedure, the mail can be removed and processed (sorted, etc.) in the usual fashion.

### EXAMPLE 2

(1) A spore impermeable removable mailbox liner/transport/treatment module is deployed in substantially all USPS mailboxes.

(2) On opening the mailbox, the module is manually or automatically sealed, such that any spores that might be in the articles of mail are safely contained therein.

(3) The sealed module is transported to a processing facility where (if, prior to or during transport, a separate humidification step has not been taken) water vapor and a sterilizing concentration of chlorine dioxide gas are injected into the module, e.g., through a septum port, and, where the chlorine dioxide gas is then allowed to reside in the module for a period of time; followed by purging of the chlorine dioxide gas from the module. The humidification and gas-exposure steps may be effected simultaneously or sequentially but humidification must be done either before or during gas treatment.

(4) The contents of the module are purged or allowed to “off-gas”, so that any residual sterilizing gas which may have been sorbed by the mail has diminished to levels to which human exposure is acceptable.

(5) After a decontamination/off-gassing procedure, the mail can be removed from the module and processed (sorted, etc.) in the usual fashion.

In order to take advantage of the time it takes to transport the mail from a consolidation facility to a processing/distribution center, humidification and gas decontamination of the mailbox liner/transport/treatment module may be accomplished in the vehicle (e.g., trailer) used to transport the mail contained in the mailbox liner/treatment module. In this embodiment, the sterilizing gas mixture is introduced into a sealed, gas tight van or truck body containing suitable mailbox modules.

In order to give an immediate visual indication that the module has been processed, the mailbox liner/transport/treatment module may be imprinted with an ink or dye which reacts with the decontaminating gas.

### EXAMPLE 3

(1) A spore-impermeable, removable opaque mailbox liner/transport/treatment module is deployed in USPS mailboxes.

(2) When the mailbox is opened, the module is manually or automatically sealed, such that any spores that might be in the articles of mail are safely contained therein.

(3) A chlorine dioxide-generating component within the sealed container, e.g., crushable ampoules of an inorganic acid and sodium chlorite in a gas-permeable packet, is activated to release chlorine dioxide gas into the interior of the module. Similarly, a wet sponge or the like may be used to provide a humid atmosphere within the module. The sealed module, which now contains mail, moisture and a sterilizing amount of chlorine dioxide gas, is transported to a processing facility where the remaining chlorine dioxide gas, if any, is purged from the module.

(4) After the decontamination procedure, the mail can be removed from the module and processed (sorted, etc.) in the usual fashion.

In order to take advantage of the time required to transport the mail from a consolidation facility to a processing/distribution center, humidification and gas decontamination of the gas-permeable mailbox liner/transport/treatment module of Example 1 may be accomplished in the vehicle (e.g., trailer) used to transport the mail. Similarly, the chlorine dioxide-generating component and humidification source of Example 3, by being activated early in the collection routine, takes advantage of almost all of the time that the module is in transit.

In circumstances where humidification and chlorine dioxide introduction are accomplished in separate steps, it may be advantageous to apply chlorine dioxide gas that contains less than 50% relative humidity, in order to avoid problems associated with condensation of water from the chlorine dioxide gas, which can adversely affect gas introduction and also cause collateral damage to the materials being treated.

In order to give an immediate visual indication that the module has been processed, mailbox liner/transport/treatment module may be imprinted with ink or dye which reacts with the decontaminating gas once sterilizing parameters (e.g., concentration×time, humidity) have been met.

While the present invention has been described and illustrated as a process for decontamination of mail, the processes of the present invention can be used to decontaminate any bio-contaminated material that must be collected and transported, such as infectious (“red bag”) waste generated in hospitals and physicians offices.

Having thus described our invention what is desired to be secured by Letters Patent of the United States is set forth in the appended claims.

What is claimed:

1. A method for decontaminating articles such as letters and packages deposited with, collected by and forwarded by one of the federal postal service or a private delivery service comprising the steps of:
collecting said articles in a receptacle that is substantially impermeable to micro-organisms but permeable to gas, and adapted to be sealed and transported; and
exposing said receptacle to an atmosphere consisting of a mixture of a sterilizing gas and a non-reactive diluent, said mixture permeable to said container, with said sterilizing gas present in an amount sufficient to decontaminate said articles.

2. A method according to claim 1 including the step of humidifying said articles in said receptacle prior to transport.

3. A method according to claim 1 including the step of humidifying said articles in said receptacle during transport.

4. A method according to claim 1 including the step of humidifying said articles in said receptacle after transport and prior to exposure to said sterilizing gas.

5. A method according to claim 1 including the step of simultaneously humidifying said articles in said receptacle and exposing said articles to said sterilizing gas.

6. A method according to claim 1 including the step of selecting parameters of sterilizing gas concentration and relative humidity of said sterilizing gas and diluent mixture and time of exposure of said articles to achieve greater than six logs of inactivation of weaponized spores including, but not limited to Anthrax and non-pathogenic surrogate of Anthrax.

7. A method according to claim 6 including the step of selecting said parameters to achieve greater than 8 logs of inactivation of said weaponized spores.

8. A method according to claim 1 including the step of using chlorine dioxide as said sterilizing gas.

9. A method according to claim 8 including the step of using chlorine dioxide gas, containing less than 0.1% chlorine gas as a contaminant.

10. A method according to claim 8 including the step of using chlorine dioxide gas containing less than 50% relative humidity.

11. A method according to claim 8 including the step of having said chlorine dioxide gas present in a concentration of up to 10,000 parts per million by volume of said mixture.

12. A method according to claim 1 including the step of using a receptacle that is also opaque to incident and ultra-violet light.

13. A method according to claim 1 including the step of using a receptacle that is substantially gas impermeable, said receptacle including means for introducing sterilizing gas into said receptacle.

14. A method according to claim 1 including the step of fabricating said receptacle from a spun-bonded polyolefin.

15. A method according to claim 8 including the step of preparing said sterilizing gas mixture by diluting chlorine dioxide gas in one of air, nitrogen, or other compatible diluent gas.

16. A method according to claim 8 including the step of using said chlorine dioxide at room temperature.

17. A method according to claim 8 including the step of using said chlorine dioxide and with said receptacle being substantially opaque to incident light.

18. A method according to claim 8 including the step of decontamination with chlorine dioxide gas in a vacuum vessel.

19. A method according to claim 1 including carrying out the method in a gas-tight vehicle used to transport said receptacle.

20. A method according to claim 1 including the step of creating an atmosphere of at least 60% relative humidity within said receptacle in advance of treatment with sterilizing gas.

21. A method according to claim 1 including the step of establishing a temperature of at least 60° F. within said receptacle prior to exposing said receptacle to sterilizing gas.

22. A method according to claim 6 including the step of exposing said articles to said sterilizing gas whereby at least a 6-log kill of microorganisms, as evidenced by standard bio-indicators, such as those containing spores of bacillus subtilus.

23. A method according to claim 7 including the step of exposing said articles to said sterilizing gas whereby at least a 8-log kill of microorganisms, as evidenced by standard bio-indicators, such as those containing spores of bacillus subtilus.

24. A method according to claim 1 including the step of exposing said articles to said sterilizing gas whereby at least a 10-log kill of microorganisms, as evidenced by standard bio-indicators, such as those containing spores of bacillus subtilus, is achieved.

25. A method according to claim 1 including the step of exposing said articles to said sterilizing gas under conditions whereby at least a 6-log kill of weaponized microorganisms, as evidenced by bio-indicators comprising specially prepared non-pathogenic surrogates of said weaponized pathogenic spores is achieved.

26. A method according to claim 1 including the step of exposing said articles to said sterilizing gas under conditions whereby at least a 8-log kill of weaponized microorganisms, as evidenced by bio-indicators comprising specially prepared non-pathogenic surrogates of said weaponized pathogenic spores is achieved.

27. A method according to claim 1 including the step of exposing said articles to said sterilizing gas under conditions whereby at least a 10-log kill of weaponized microorganisms, as evidenced by bio-indicators comprising specially prepared non-pathogenic surrogates of said weaponized pathogenic spores is achieved.

28. A system for collecting articles such as letters and packages deposited with, collected by and forwarded by one of the federal postal service or private delivery service companies comprising in combination:
a receptacle adapted to receive said articles said receptacle being substantially impermeable to micro-organisms and permeable to a sterilizing gas, and
means to seal said receptacle in order to contain said articles within said receptacle for transport.

29. A system according to claim 28 wherein said receptacle is a bag.

30. A system according to claim 28 wherein said receptacle is a rigid wall container.

31. A system according to claim 28 wherein said receptacle is a opaque to incident light.

32. A system according to claim 28 wherein said receptacle is fabricated from a spun-bonded polyolefin.

33. A system according to claim 28 including means to inject and remove sterilize gas from said receptacle.

34. A system according to claim 28 including a user-activated chlorine dioxide releasing means in said container.

35. A system according to claim 28 including a moisture activated chlorine dioxide releasing means in said container.

36. A system according to claim 28 including indication means in said receptacle to indicate decontamination of said container has been completed.

37. A method for decontaminating bio-contaminated articles comprising the step of:
placing said articles in one of a receptacle that is substantially impermeable to micro-organisms but permeable to gas or substantially impermeable to both micro-organisms and gas; and
exposing said articles to an atmosphere consisting of a mixture of a sterilizing gas and a diluent, said mixture containing an effective amount of sterilizing gas to decontaminate said bio-contaminated articles.

38. A method according to claim 37 including the step of humidifying said articles in said receptacle prior to transport.

39. A method according to claim 37 including the step of humidifying said articles in said receptacle during transport.

40. A method according to claim 37 including the step of humidifying said articles in said receptacle after transport and prior to exposure to said sterilizing gas.

41. A method according to claim 37 including the step of simultaneously humidifying said articles in said receptacle and exposing said articles to said sterilizing gas.

42. A method according to claim 37 including the step of selecting parameters of sterilizing gas concentration and relative humidity of said sterilizing gas and diluent mixture and time of exposure of said articles to achieve greater than six logs of inactivation of weaponized spores including, but not limited to Anthrax and a non-pathogenic surrogate of Anthrax.

43. A method according to claim 37 including the step of selecting said parameters to achieve greater than 8 logs of inactivation of said weaponized spores.

44. A method according to claim 37 including the step of using chlorine dioxide as said sterilizing gas.

45. A method according to claim 37 including the step of using chlorine dioxide gas, containing less than 0.1% chlorine gas as a contaminant.

46. A method according to claim 37 including the step of using chlorine dioxide gas containing less than 50% relative humidity.

47. A method according to claim 37 including the step of having said chlorine dioxide gas present in a concentration of up to 10,000 parts per million by volume of said mixture.

48. A method according to claim 37 including the step of using a receptacle that is also opaque to incident and ultra-violet light.

49. A method according to claim 37 including the step of using a receptacle that is substantially gas impermeable, said receptacle including means for introducing sterilizing gas into said receptacle.

50. A method according to claim 37 including the step of fabricating said receptacle from a spun-bonded polyolefin.

51. A method according to claim 37 including the step of preparing said sterilizing gas mixture by diluting chlorine dioxide gas in one of air, nitrogen, or other compatible diluent gas.

52. A method according to claim 37 including the step of using said chlorine dioxide at room temperature.

53. A method according to claim 37 including the step of using said chlorine dioxide and with said receptacle being substantially opaque to incident light.
